(19) United States
(12) Patent Application Publication
(10) Pub. No.: US 2024/0424261 A1
(43) Pub. Date: Dec. 26, 2024

(54) Secondary Securement Device for Vascular Access Device

(71) Applicant: BECTON, DICKINSON AND COMPANY, Franklin Lakes, NJ (US)

(72) Inventors: Atharva Shetye, Bangalore (IN); Sujeendran Malayath, Bangalore (IN); Vivek K T, Bengaluru (IN); Rahul Malviya, Salmon Arm (CA); Divik Bhargava, Meerut (IN); Srimannarayana Myla, Pune (IN); Shishir Prasad, Ramsey, NJ (US); Aniket Kulkarni, Pune (IN)

(21) Appl. No.: 18/572,371

(22) PCT Filed: Jan. 3, 2023
(86) PCT No.: PCT/US23/10023

§ 371 Date: Dec. 20, 2023

(51) Int. Cl. A61M 25/02; A61M 39/02

(52) U.S. Cl. CPC A61M 25/02; A61M 39/0247; A61M 2025/0266; A61M 2025/028; A61M 2039/0258; A61M 2205/0216

(57) ABSTRACT

A secondary securement device for a vascular access device having tubing includes a base including a first end, a second end positioned opposite the first end, a first side, a second side positioned opposite the first side, a top surface, and a bottom surface positioned opposite the top surface, with the bottom surface configured to be in contact with a patient's skin surface, and a plurality of pegs connected to the base and extending from the top surface of the base. Adjacent pegs of the plurality of pegs are configured to secure the tubing of the vascular access device.

### BACKGROUND OF THE INVENTION

### Field of the Invention

The present disclosure relates to a secondary securement device for a vascular access device.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal. Common types of VADs are an over-the-needle peripheral intravenous catheter (PIVC) or central venous catheter (CVC). A variety of solutions exist for securing VADs and maintaining the insertion site; however, clinicians are often left with limited solutions to manage the portions of the lumen that extend outside of the body. These loose extensions can create opportunities for the lumens to be pulled, potentially compromising the catheter position or insertion site integrity. Tape and textile dressings are sometimes utilized, but can be cumbersome when the line needs to be accessed. In addition, while a line is being accessed, an un-capped line is at risk for touch contamination as the clinician often needs to perform multiple steps.

### SUMMARY OF THE INVENTION

In one aspect or embodiment, a secondary securement device for a vascular access device having tubing includes a base including a first end, a second end positioned opposite the first end, a first side, a second side positioned opposite the first side, a top surface, and a bottom surface positioned opposite the top surface, with the bottom surface configured to be in contact with a patient's skin surface, and a plurality of pegs connected to the base and extending from the top surface of the base, where adjacent pegs of the plurality of pegs are configured to secure the tubing of the vascular access device.

The plurality of pegs may be arranged into rows and columns. Each of the plurality of pegs may be engaged with at least one other of the plurality of pegs. At least a portion of the plurality of pegs may be arranged in pairs. The plurality of pegs may be spaced from each other. Each of the plurality of pegs may include a connecting portion connected to the base and a head portion extending from the connecting portion. The head portion may be semi-spherical or spherical. The connecting portion of each peg may include a recessed area configured to receive a portion of the tubing of the vascular access device. The secondary securement device may further include first and second anchors configured to be secured to the tubing of the vascular access device. The plurality of pegs may include an elastomeric material. The bottom surface of the base may include an adhesive surface and a liner.

In a further aspect or embodiment, a secondary securement device for a vascular access device having tubing includes a base including a first end, a second end positioned opposite the first end, a first side, a second side positioned opposite the first side, a top surface, and a bottom surface positioned opposite the top surface, with the bottom surface configured to be in contact with a patient's skin surface, a first securing member connected to the top surface of the base, with the first securing member including a first surface configured to receive the tubing of the vascular access device at a first location, and a second securing member connected to the top surface of the base, with the second securing member including a second surface configured to receive the tubing of the vascular access device at a second location. The first securing member is positioned closer to the first side of the base than the second securing member.

The first surface and the second surface may each be channels configured to receive the tubing of the vascular access device. The first and second securing members may be C-shaped. The first surface may face the first side of the base, and the second surface may face the second side of the base. The first securing member may be positioned closer to the first end of the base than the second securing member. The base and the first and second securing members may be formed integrally as one piece. The secondary securement device may include a third securing member connected to the top surface of the base, with the third securing member including a first surface configured to receive the tubing of the vascular access device at a third location. The first and second securing members may include an elastomeric material.

In a further aspect or embodiment, a secondary securement device for a vascular access device having tubing includes a base including a first end, a second end positioned opposite the first end, a first side, a second side positioned opposite the first side, a top surface, and a bottom surface positioned opposite the top surface, with the bottom surface configured to be in contact with a patient's skin surface, a plurality of first connection interfaces positioned on the top surface of the base, and a securing member configured to secure the tubing of the vascular access device, with the securing member including a second connection interface configured to be connected to at least one of the plurality of first connection interfaces.

The plurality of first connection interfaces may each be an opening, and the second connection interface may each be a protrusion configured to be received by one of the openings. The securing member may include first and second clips, each configured to receive and secure the tubing of the vascular access device. The second connection interface may be spaced from the first and second clips.

The plurality of first connection interfaces may each be a protrusion, and the second connection interface is an opening configured to receive one or more of the protrusions. At least one of the protrusions of the plurality of first connection interfaces may be configured to engage internal structure of the securing member to secure the securing member to the base. The securing member may include a clip configured to receive and secure the tubing of the vascular access device.

The bottom surface of the base may be arched. The base may be flexible and configured to conform to a skin surface of a patient.

### BRIEF DESCRIPTION OF THE DRAWINGS

FIG. 1 is a perspective view of a secondary securement device for a vascular access device according to one aspect or embodiment of the present application;

FIG. 2 is a top view of the secondary securement device of FIG. 1;

FIG. 3 is a front view of the secondary securement device of FIG. 1;

FIG. 4 is a cross-sectional view of the secondary securement device of FIG. 1;

FIG. 5 is a top view of the secondary securement device of FIG. 1, showing the secondary securement device in use;

FIG. 6 is a perspective view of a vascular access device in use according to one aspect or embodiment of the present application;

FIG. 7 is a perspective view of the secondary securement device of FIG. 1, showing the secondary securement device secured to tubing of a vascular access device;

FIG. 8 is a cross-sectional view of the secondary securement device of FIG. 1, showing tubing of a vascular access device engaged with the secondary securement device;

FIG. 9 is a cross-sectional view of the secondary securement device of FIG. 1, showing tubing of a vascular access device secured with the secondary securement device;

FIG. 10 is a perspective view of the secondary securement device of FIG. 1, showing the secondary securement device secured to tubing of a vascular access device;

FIG. 11 is a perspective view of a secondary securement device for a vascular access device according to a further aspect or embodiment of the present application;

FIG. 12 is a perspective view of a secondary securement device for a vascular access device according to a further aspect or embodiment of the present application;

FIG. 13 is a perspective view of the secondary securement device of FIG. 1, showing a liner being removed;

FIG. 14 is a perspective view of the secondary securement device of FIG. 11, showing the secondary securement device in use;

FIG. 15 is a perspective view of a vascular access device according to one aspect or embodiment of the present application, showing an anchor positioned on tubing of the vascular access device;

FIG. 16 is a top view of the secondary securement device of FIG. 11, showing the secondary securement device secured to tubing of a vascular access device;

FIG. 17 is a perspective view of the secondary securement device of FIG. 11, showing the secondary securement device secured to tubing of a vascular access device;

FIG. 18 is a perspective view of the secondary securement device of FIG. 11, showing the secondary securement device secured to multiple lines of a vascular access device;

FIG. 19 is a perspective view of a secondary securement device for a vascular access device according to a further aspect or embodiment of the present application;

FIG. 20 is a perspective view of the secondary securement device of FIG. 19;

FIG. 21 is a perspective view of the secondary securement device of FIG. 19, showing the secondary securement device in use;

FIG. 22 is a perspective view of a secondary securement device for a vascular access device according to a further aspect or embodiment of the present application;

FIG. 23 is a perspective view of a securing member of the secondary securement device of FIG. 22;

FIG. 24 is a top view of the secondary securement device of FIG. 22, showing tubing of a vascular access device being positioned next to the secondary securement device;

FIG. 25 is a top view of the secondary securement device of FIG. 22, showing tubing of a vascular access device being secured to the secondary securement device;

FIG. 26 is a top view of the secondary securement device of FIG. 22, showing tubing of a vascular access device secured to the secondary securement device;

FIG. 27 is a top view of the secondary securement device of FIG. 22, showing tubing and additional components of a vascular access device secured to the secondary securement device;

FIG. 28 is a perspective view of a secondary securement device for a vascular access device according to a further aspect or embodiment of the present application;

FIG. 29 is a perspective view of a securing member of the secondary securement device of FIG. 28; and

FIG. 30 is a perspective view of a base of the secondary securement device of FIG. 28.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

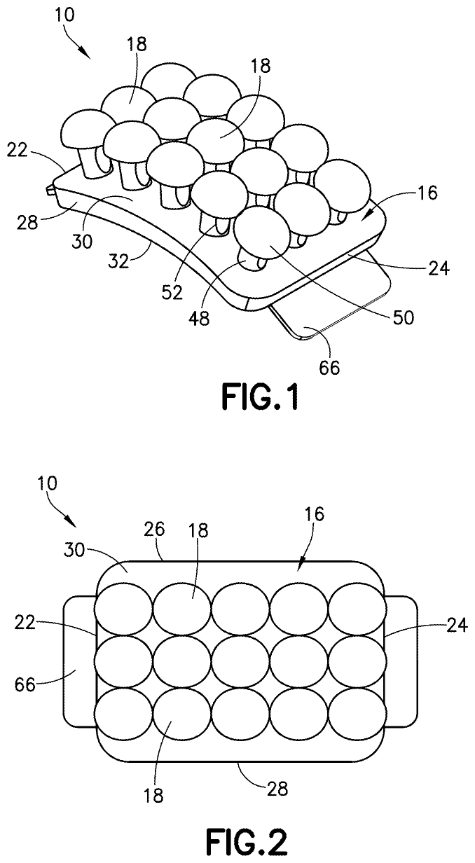

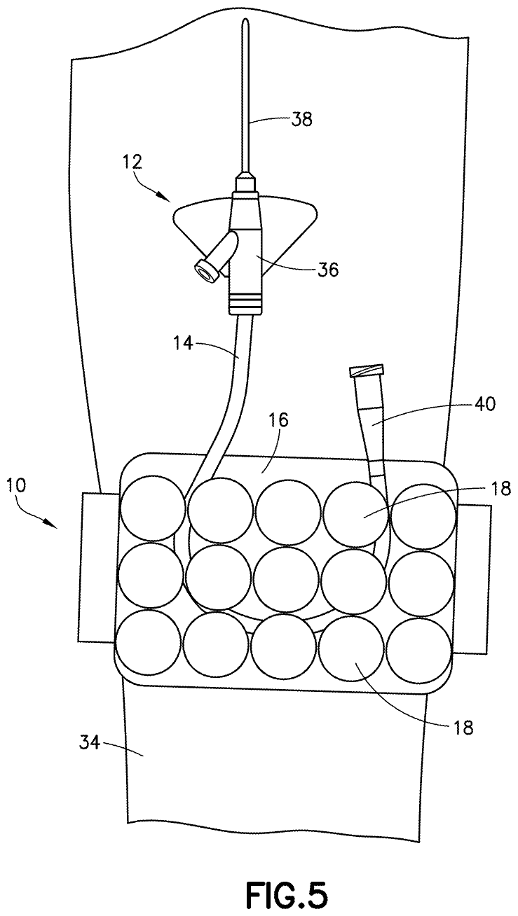

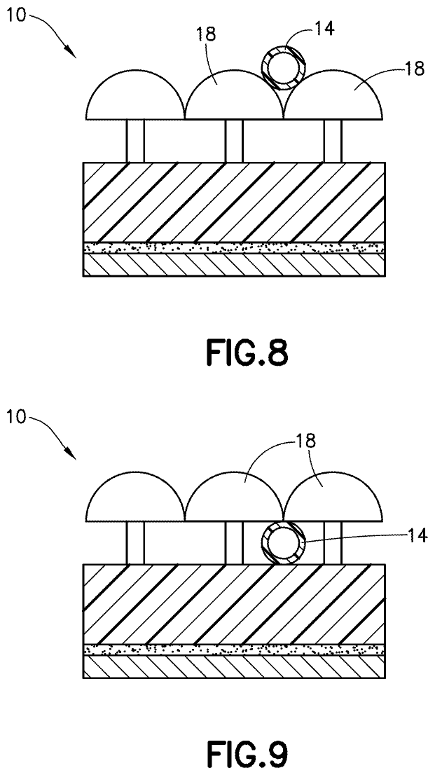

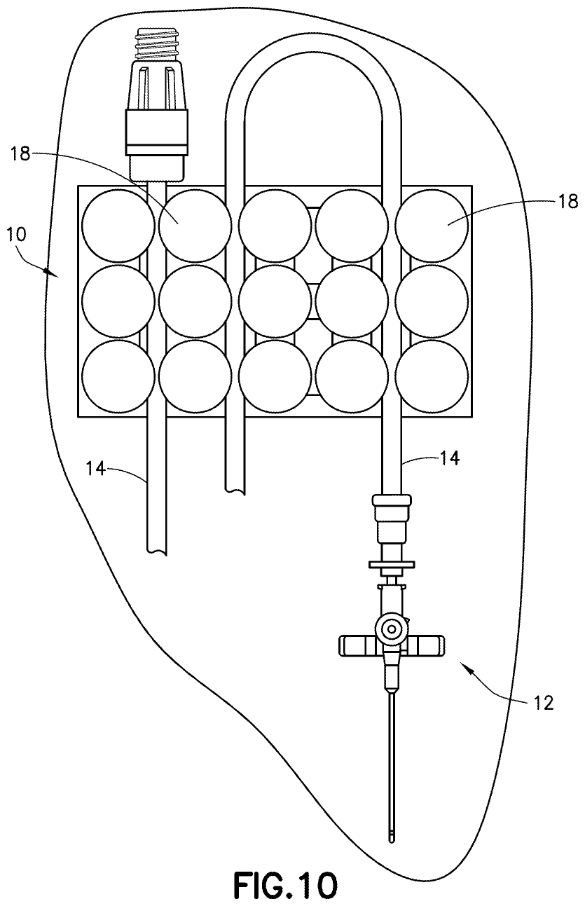

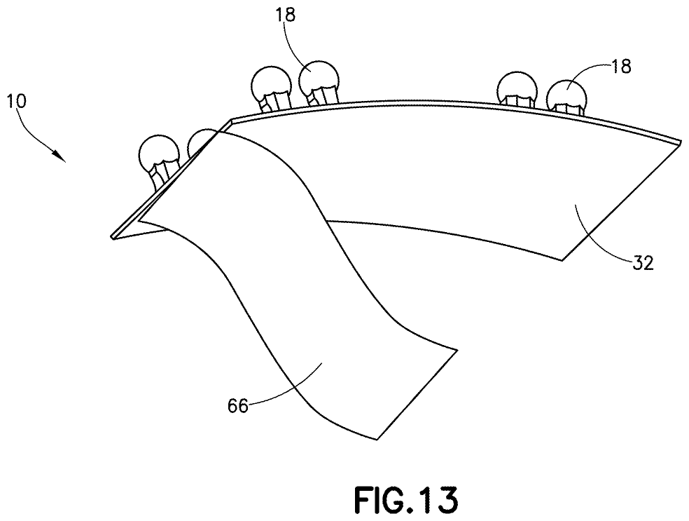

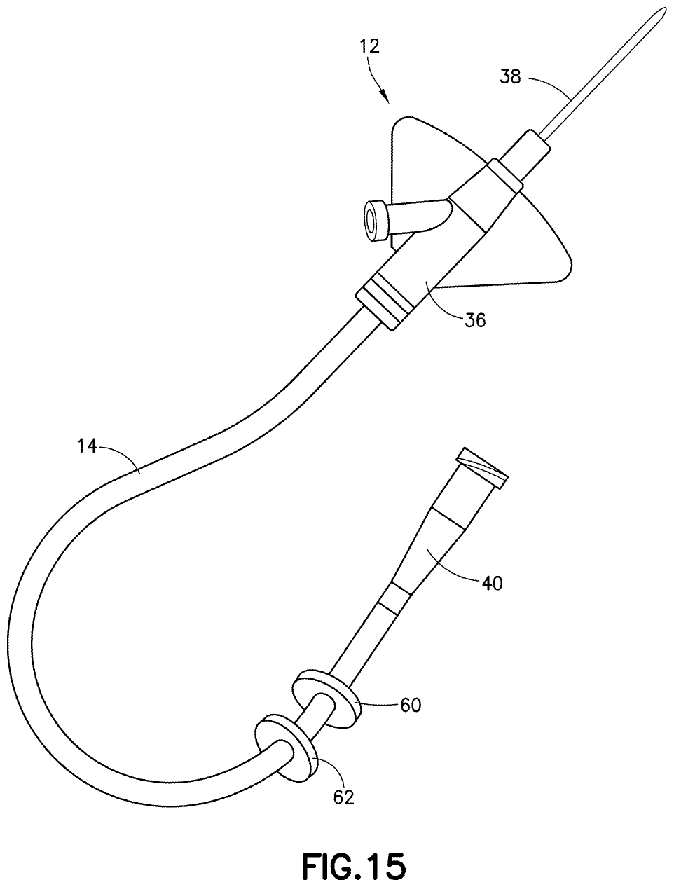

Referring to FIGS. 1-18, in one aspect or embodiment, a secondary securement device 10 for a vascular access device 12 having tubing 14 includes a base 16, and a plurality of pegs 18 connected to the base 16. The base 16 includes a first end 22, a second end 24 positioned opposite the first end 22, a first side 26, a second side 28 positioned opposite the first side 26, a top surface 30, and a bottom surface 32 positioned opposite the top surface 30, with the bottom surface 32 configured to be in contact with a patient's skin surface 34. The plurality of pegs 18 extend from the top surface 30 of the base 16. Adjacent pegs 18 of the plurality of pegs 18 are configured to secure the tubing 14 of the vascular access device 12. The plurality of pegs 18 are configured to provide multiple locations for securing the tubing 14 or other medical components, with the base 16 being secured to the skin surface 34 of the patient. In some aspects or embodiments, as shown in FIGS. 8 and 9, each of the plurality of pegs 18 are flexible to allow the tubing to be pushed between the adjacent pegs 18 of the plurality of pegs 18 until the tubing 14 engages the base 16, with the pegs 18 securing the tubing 14 between the pegs 18.

As discussed in additional detail below, the secondary securement device 10 is configured to hold and secure the tubing 14 of the vascular access device 12 to prevent accidental pull forces from transmitting to the vascular access device 12 and placed catheter. The vascular access device 12 may be a peripheral intravenous catheter or central venous catheter, although the secondary securement device 10 may be utilized in connection with other medical devices, such as midline catheters, urinary catheters, enteral catheters, and dialysis catheters. In some aspects or embodiments, the vascular access device 12 includes a catheter adapter 36, a catheter 38, the tubing 14, and a connector 40 positioned at a proximal end of the tubing 14.

Referring to FIGS. 1-10, in one aspect or embodiment, the plurality of pegs 18 are arranged into rows and columns. As shown, the plurality of pegs 18 are arranged in a 3×5 array, although other suitable arrays, such as 2×2, 2×3, 3×3, 3×4, etc., may be utilized. As shown in FIG. 4, for example, each of the plurality of pegs 18 may be engaged with at least one other of the plurality of pegs 18. Alternatively, the plurality of pegs 18 may be positioned sufficiently close to each other to allow the tubing 14 to be pushed between two pegs 18 while inhibiting removal of the tubing 14 once pushed between the pegs 18.

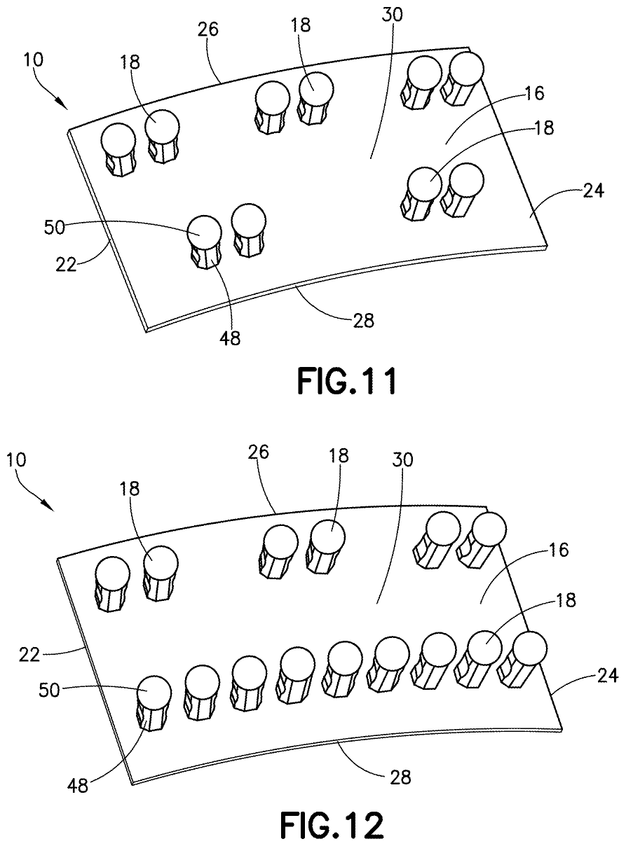

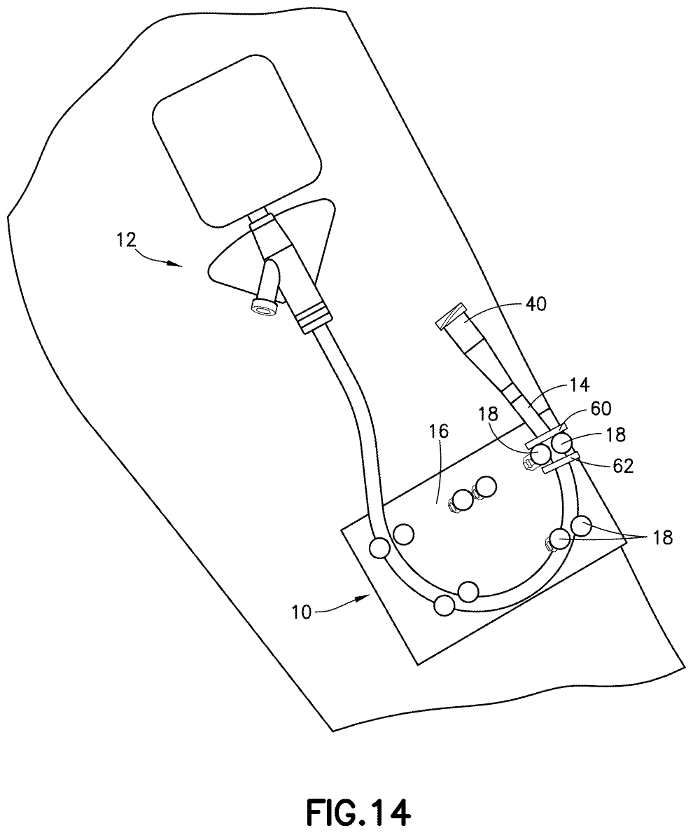

Referring to FIGS. 11-18, in one aspect or embodiment, at least a portion of the plurality of pegs 18 are arranged in pairs. As shown in FIG. 11, five pairs of pegs 18 are provided on the top surface 30 of the base 16, with the pairs of pegs 18 spaced from each other, although one or more pairs of pegs 18 and other configurations may be provided. As shown in FIG. 12, three pairs of pegs 18 are provided near the first side 26 of the base 16, with a row of pegs 18 provided near the second side 28 of the base 16. The plurality of pegs 18 are spaced from each other, although one or more of the pegs 18 may be engaged with each other. As shown in FIG. 14, for example, the tubing 14 of the vascular access device 12 may be positioned between adjacent pegs 18 to hold and secure the tubing 14 and allow for various positions and routing of the tubing 14.

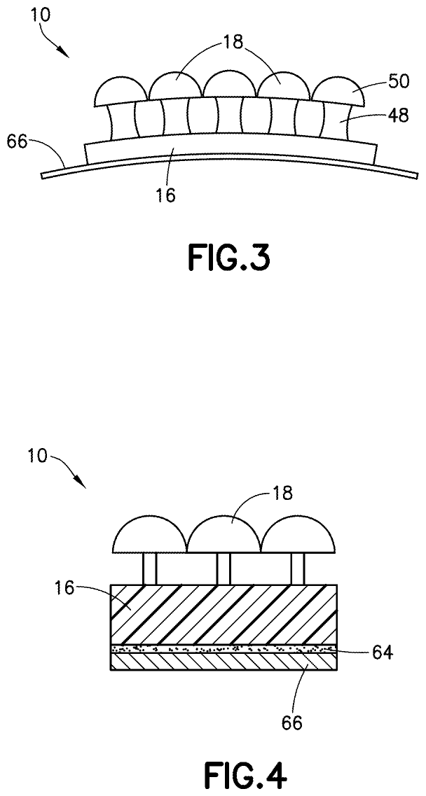

Referring to FIGS. 3, 4, and 17, each of the plurality of pegs 18 includes a connecting portion 48 connected to the base 16 and a head portion 50 extending from the connecting portion 48. As shown in FIGS. 3 and 4, in some aspects or embodiments, the head portion 50 is semi-spherical. As shown in FIG. 17, in some aspects or embodiments, the head portion 50 is spherical. The head portion 50 is wider than the connecting portion 48 to allow the tubing 14 or other component to be pushed between adjacent head portions 50 to be seated between the adjacent connecting portions 48. The connecting portion 48 of each peg 18 includes a recessed area 52 configured to receive a portion of the tubing 14 of the vascular access device 12. The recessed area 52 may be provided to create more space for the tubing 14 or other component of the vascular access device 12 and/or to provide additional surface contact for griping the tubing 14 or other component.

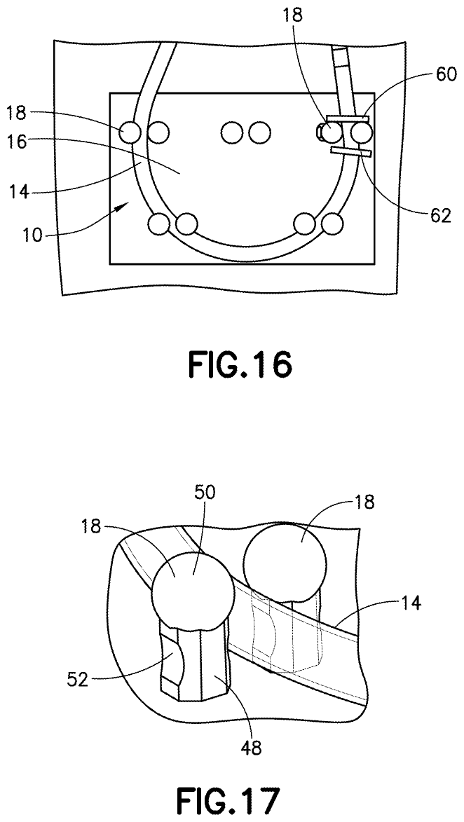

Referring to FIGS. 14-16, the secondary securement device 10 may include first and second anchors 60, 62 configured to be secured to the tubing 14 of the vascular access device 12. The first and second anchors 60, 62 are spaced from each other and may be secured to the tubing 14 prior to positioning of the tubing 14 between the pegs 18 or after positioned between the pegs 18. The first and second anchors 60, 62 may are disc-shaped, although the first and second anchors 60, 62 may include an opening (not shown) to allow the anchors 60, 62 to be positioned over the tubing 14. As shown in FIG. 16, with the tubing 14 positioned between adjacent pegs 18, the first and second anchors 60, 62 may be positioned on opposite sides of the pegs 18 to prevent or inhibit axial movement of the tubing 14.

In some aspects or embodiments, the base 16 and/or the plurality of pegs 18 include an elastomeric material. The base 16 and/or the plurality of pegs 18 may be molded together from a rubber or silicone material. The bottom surface 32 of the base 16 may be curved or arched and configured to conform to the skin surface 34 of the patient. The bottom surface 32 of the base 16 includes an adhesive surface 64 and a liner 66, with the liner 66 configured to be peeled or removed and the adhesive surface 64 securing the base 16 to the skin surface 34 of the patient.

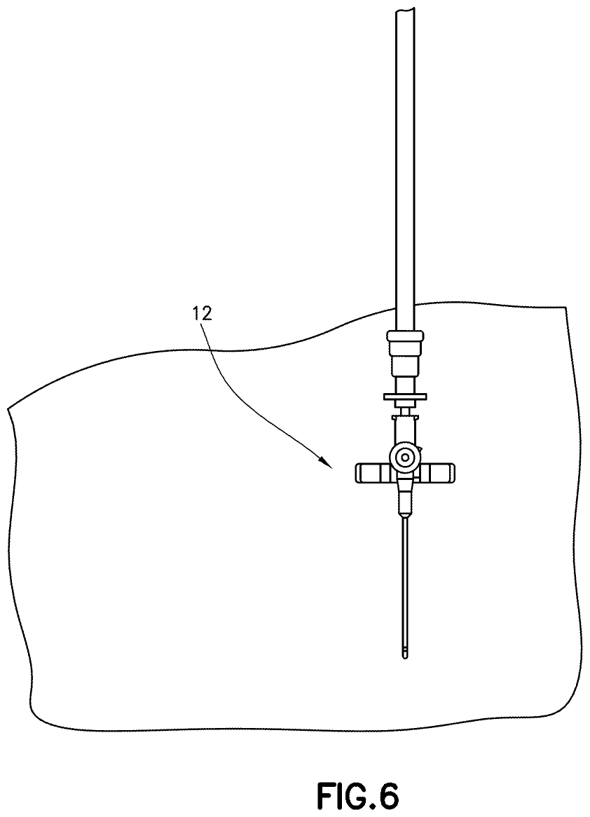

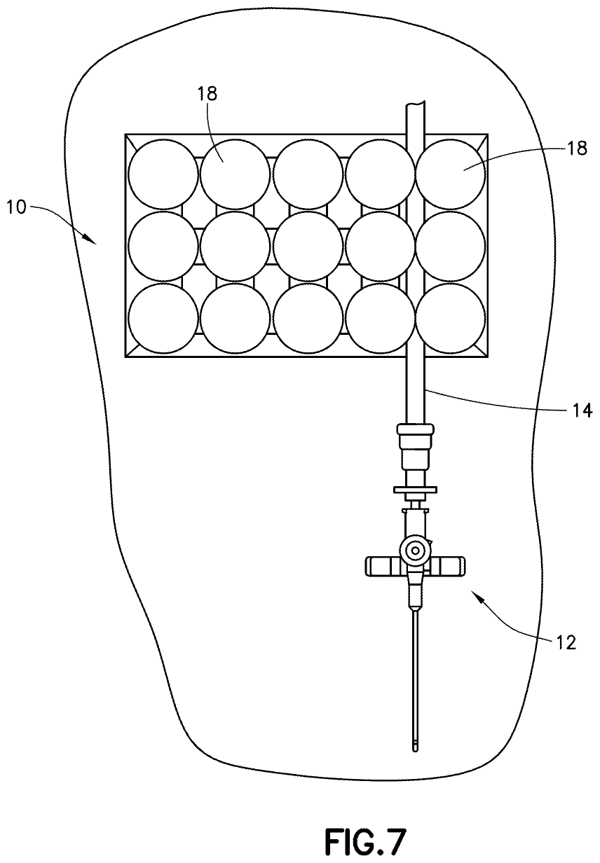

Referring to FIGS. 6-10, after placing the catheter 38 of the vascular access device 12 pursuant to clinical procedure, the base 16 of the secondary securement device 10 is attached to the skin surface 34 of the patient via the adhesive surface 64 on the bottom surface 32 of the base 16 or other suitable arrangement. As shown in FIGS. 7-9, the tubing 14 is pushed between adjacent pegs 18 until the tubing 14 is between the head portion 50 and/or in contact with the top surface 30 of the base 16, with the pegs 18 securing and retaining the tubing 14 or other component of the vascular access device 12 to the base 16. As shown in FIG. 10, the tubing 14 is formed into a J-shaped or inverse J-shaped loop with another portion of the tubing 14 secured to the secondary securement device 10 in the same manner as described above. Additional tubing and/or further components may also be separately secured to the secondary securement device 10 in the same manner. The plurality of pegs 18 are configured to secure and hold tubing 14 having various outer diameters.

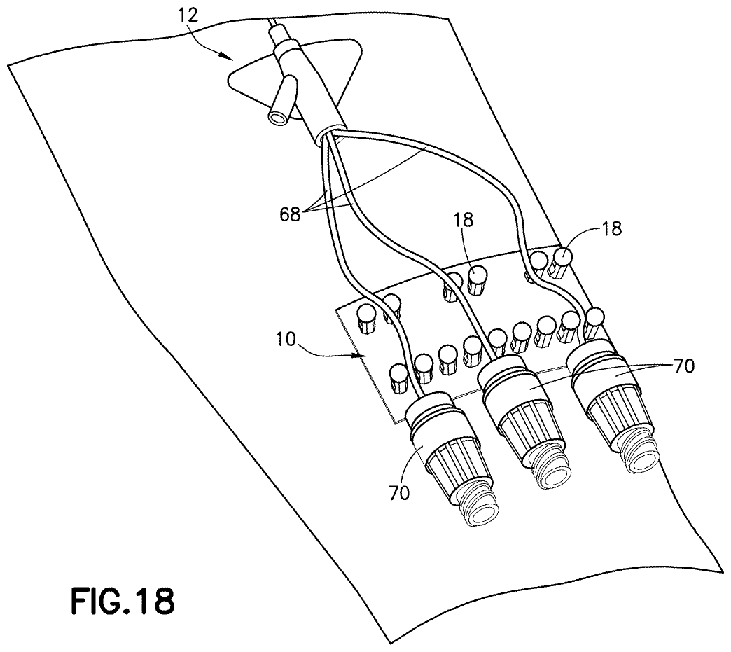

Referring to FIGS. 14-18, in a similar manner as described above in connection with FIGS. 6-10, the tubing 14 is positioned between the pairs of pegs 18 and is formed in to a J-shaped or inverse J-shaped loop, with the first and second anchors 60, 62 positioned on opposite sides of two or more pegs 18 to prevent or inhibit axial movement of the tubing 14. Referring to FIG. 18, in some aspects or embodiments, the plurality of pegs 18 are configured to hold and secure multiple lines 68 with multiple medical connectors 70, such as needle-free connectors (NFC). The different NFC positions can be marked and identified with different treatment plans or medications for the patient for easy NFC assignment and identification present near the patient.

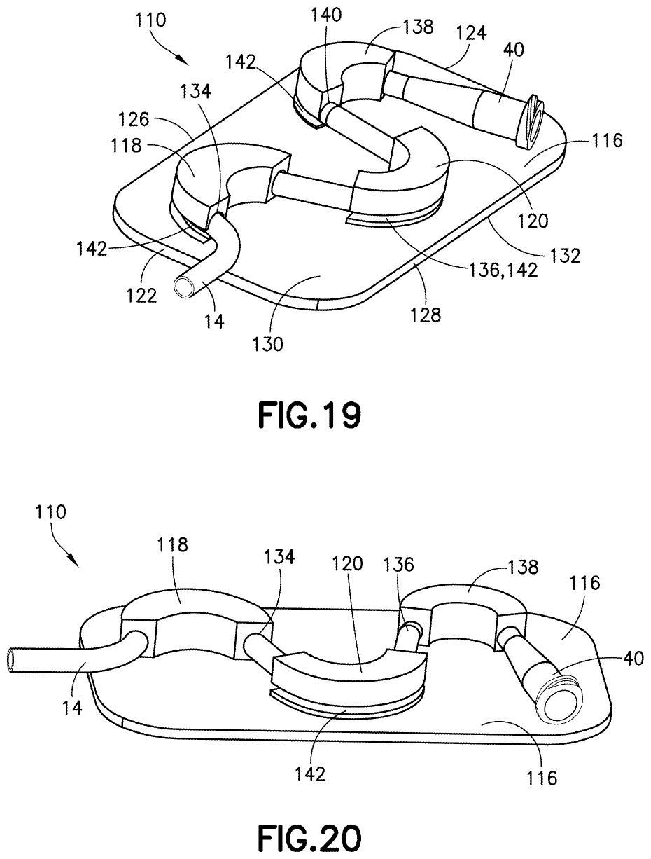

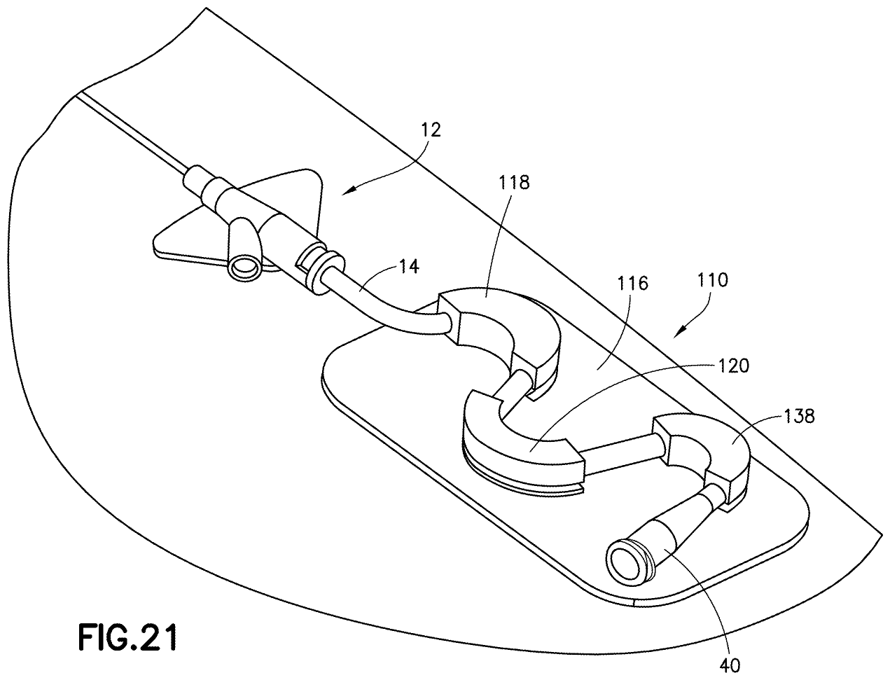

Referring to FIGS. 19-21, in a further aspect or embodiment, a secondary securement device 110 for the vascular access device 12 includes a base 116, a first securing member 118 connected to the base 116, and a second securing member 120 connected to the base 116. The base 116 includes first end 122, a second end 124 positioned opposite the first end 122, a first side 126, a second side 128 positioned opposite the first side 126, a top surface 130, and a bottom surface 132 positioned opposite the top surface 130, with the bottom surface 132 configured to be in contact with the patient's skin surface 34. The first securing member 118 includes a first surface 134 configured to receive the tubing 14 of the vascular access device 12 at a first location and the second securing member 120 includes a second surface 136 configured to receive the tubing 14 of the vascular access device 12 at a second location. The first securing member 118 is positioned closer to the first side 126 of the base 116 than the second securing member 120. The first and second securing members 118, 120 are configured to hold and secure the tubing 14 of the vascular access device 12 to prevent accidental pull forces from transmitting to the vascular access device 12 and placed catheter 38.

Referring again to FIGS. 19-21, the first surface 134 and the second surface 136 are each channels configured to receive the tubing 14 of the vascular access device 12. The first and second securing members 118, 120 and respective first and second surfaces 134, 136 are C-shaped. The first surface 134 faces the first side 126 of the base 116 and the second surface 136 faces the second side 128 of the base 116. The first securing member 118 is positioned closer to the first end 122 of the base 116 than the second securing member 120. In some aspects or embodiments, the base 116 and the first and second securing members 118, 120 are formed integrally as one piece. The secondary securement device 110 may be molded from an elastomeric material, such as rubber or silicone, although the first and second securing members 118, 120 may be formed separately and attached to the base 116. The secondary securement device 110 also includes a third securing member 138 connected to the top surface 130 of the base 116, with the third securing member 138 including a third surface 140 configured to receive the tubing 14 of the vascular access device 12 at a third location. In some aspects or embodiments, the first, second, and third surfaces 134, 136, 140 are shaped and sized to conform to the tubing 14, with a slit 142 defined by the securing members 118, 120, 138 allowing the tubing 14 to be engaged with the surfaces 134, 136, 140. In other words, the securing members 118, 120, 138 are flexible to allow the tubing 14 to be pushed through the slit 142 to be engaged by the surfaces 134, 136, 140 thereby securing the tubing 14 to the base 116. As shown, the tubing 14 is configured to be arranged in a sinusoidal pattern, although the securing members 118, 120, 138 may be arranged to allow any other suitable position of the tubing 14. The bottom surface 132 of the base 116 may include an adhesive surface and/or liner (not shown) to enable the base 116 to be secured to the skin surface 34 of the patient.

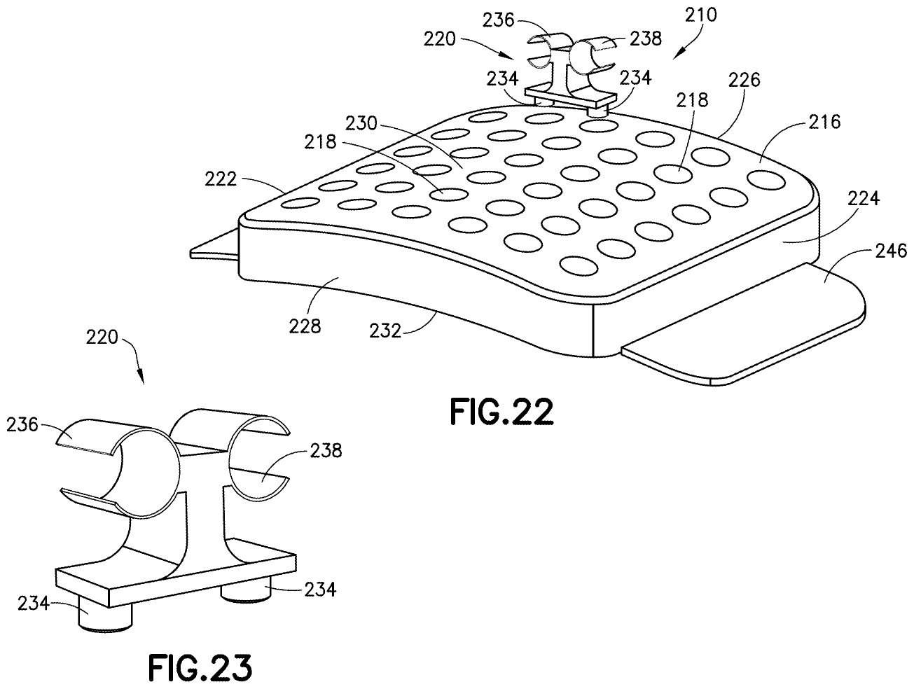

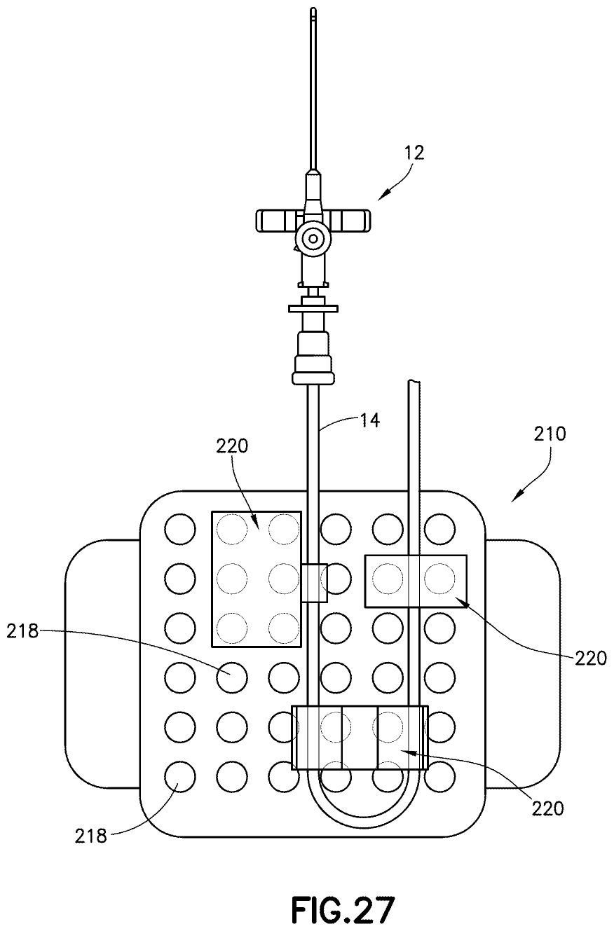

Referring to FIGS. 22-30, in a further aspect or embodiment, a secondary securement device 210 for the vascular access device 12 includes a base 216, a plurality of first connection interfaces 218, and a securing member 120. The base 216 includes a first end 222, a second end 224 positioned opposite the first end 222, a first side 226, a second side 228 positioned opposite the first side 226, a top surface 230, and a bottom surface 232 positioned opposite the top surface 230, with the bottom surface 232 configured to be in contact with the patient's skin surface 34. The plurality of first connection interfaces 218 are positioned on the top surface 230 of the base 216. The securing member 220 is configured to secure the tubing 14 of the vascular access device 12. The securing member 220 includes a second connection interface 234 configured to be connected to at least one of the plurality of first connection interfaces 218.

Referring to FIGS. 22 and 23, in one aspect or embodiment, the plurality of first connection interfaces 218 are openings, and the second connection interface 234 is a protrusion configured to be received by one of the openings. The securing member 220 is configured to be secured to the base 216 when the second connection interface 234 is received within one of the plurality of first connection interfaces 218. As shown in FIG. 23, the securing member 220 may include two second connection interfaces 234 each received by a respective one of the plurality of first connection interfaces 218. The securing member 220 includes first and second clips 236, 238 each configured to receive and secure the tubing 14 of the vascular access device 12. The second connection interface 234 is spaced from the first and second clips 236, 238, although other suitable arrangements may be utilized.

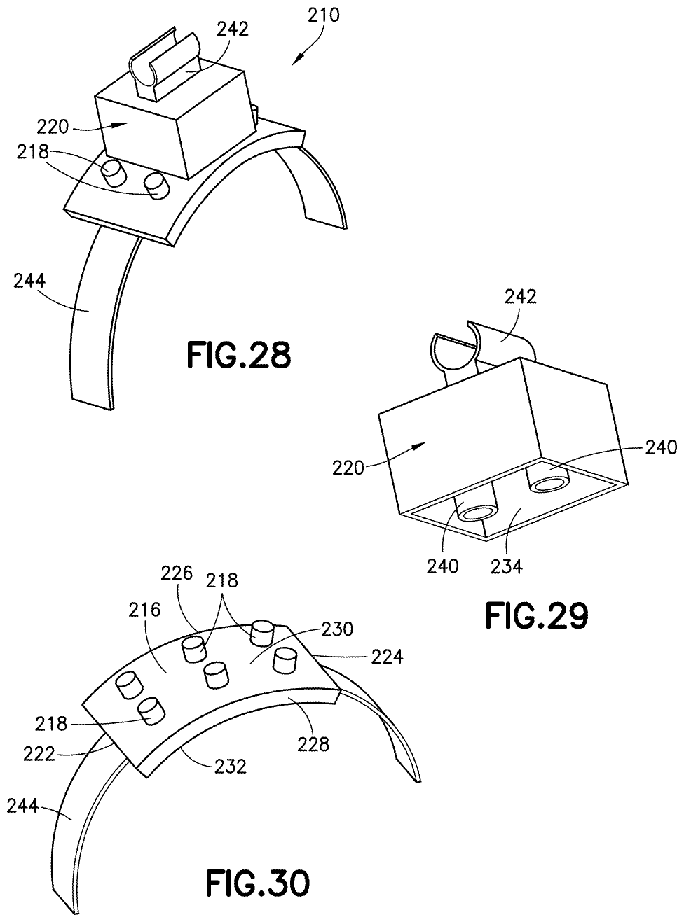

Referring to FIGS. 28-30, in one aspect or embodiment, the plurality of first connection interfaces 218 are protrusions, and the second connection interface 234 is an opening configured to receive one or more of the protrusions. In some aspects or embodiments, at least one of the first connection interfaces 218 is configured to engage internal structure 240, such as a post, of the securing member 220 to secure the securing member 220 to the base 216. The securing member 220 includes a clip 242 configured to receive and secure the tubing 14 of the vascular access device 12. The base 216 may include a band 244 for securing the base 216 to an arm or other body part of a patient.

In some aspects or embodiments, the bottom surface 232 of the base 216 is arched and/or flexible and configured to conform to the skin surface 34 of the patient. The bottom surface 232 of the base 216 may include an adhesive surface and liner 246, with the adhesive surface configured to secure the base 216 to the skin surface 34 of the patient. The plurality of first connection interfaces 218 allows the securing member 220 to be oriented in a variety of different positions. Further, one or more securing members 220 may be provided to secure the tubing 14 at multiple locations or to secure additional tubing and/or components of the vascular access device 12. The first connection interfaces 218 and the second connection interfaces 234 may be formed from a polymeric material.

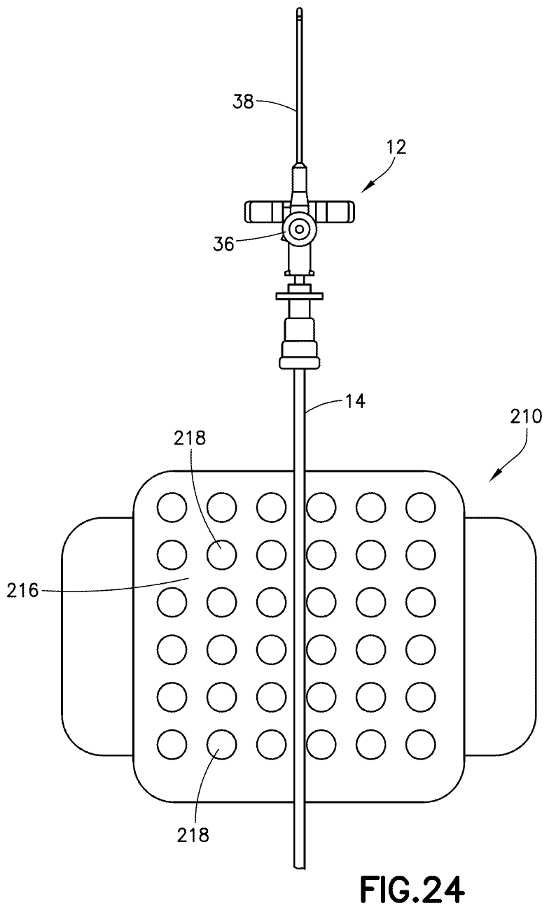

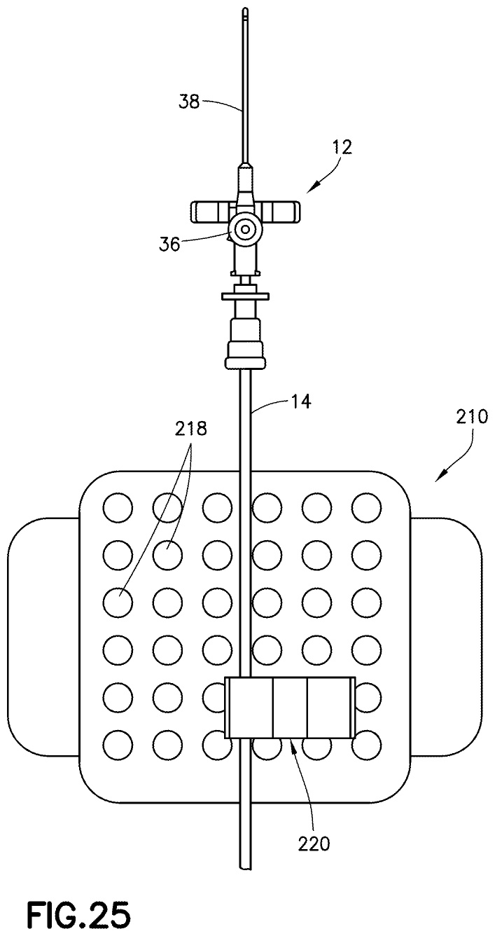

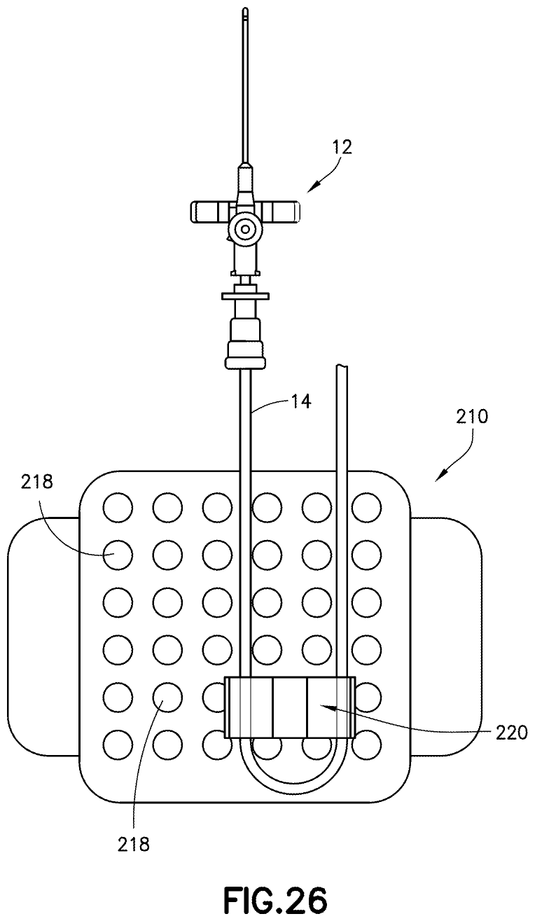

Referring to FIGS. 24-27, after placing the catheter 38 of the vascular access device 12 pursuant to clinical procedures, the base 216 is attached to the skin surface 34 of the patient via adhesive or other suitable arrangement. As shown in FIG. 25, the securing member 220 is connected to the base 216 by mating the second connection interface 234 with one or more of the first connection interfaces 218, with the tubing 14 pushed or connected to one of the clips 236, 238 of the securing member 220 to secure the tubing 14 to the base 216. As shown in FIG. 26, the tubing 14 is formed into a J-shaped or inverse J-shaped loop with another portion of the tubing 14 secured to one of the clips 236, 238 of the securing member 220. Additional tubing and/or further components may also be separately secured to the secondary securement device 210 in the same manner. The second connection interface 234 of the securing member 220 is configured to be readily connected and disconnected from the first connection interfaces 218 of the base 216.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A secondary securement device for a vascular access device comprising tubing, the secondary securement device comprising:
a base including a first end, a second end positioned opposite the first end, a first side, a second side positioned opposite the first side, a top surface, and a bottom surface positioned opposite the top surface, the bottom surface configured to be in contact with a patient's skin surface; and
a plurality of pegs connected to the base and extending from the top surface of the base, wherein adjacent pegs of the plurality of pegs are configured to secure the tubing of the vascular access device.

2. The secondary securement device of claim 1, wherein the plurality of pegs are arranged into rows and columns.

3. The secondary securement device of claim 2, wherein each of the plurality of pegs is engaged with at least one other of the plurality of pegs.

4. The secondary securement device of claim 1, wherein at least a portion of the plurality of pegs are arranged in pairs.

5. The secondary securement device of claim 4, wherein the plurality of pegs are spaced from each other.

6. The secondary securement device of claim 1, wherein each of the plurality of pegs comprises a connecting portion connected to the base and a head portion extending from the connecting portion.

7. The secondary securement device of claim 6, wherein the head portion is semi-spherical.

8. The secondary securement device of claim 6, wherein the head portion is spherical.

9. The secondary securement device of claim 6, wherein the connecting portion of each peg comprises a recessed area configured to receive a portion of the tubing of the vascular access device.

10. The secondary securement device of claim 1, further comprising first and second anchors configured to be secured to the tubing of the vascular access device.

11. The secondary securement device of claim 1, wherein the plurality of pegs comprise an elastomeric material.

12. The secondary securement device of claim 1, wherein the bottom surface of the base comprises an adhesive surface and a liner.

13. A secondary securement device for a vascular access device comprising tubing, the secondary securement device comprising:
a base including a first end, a second end positioned opposite the first end, a first side, a second side positioned opposite the first side, a top surface, and a bottom surface positioned opposite the top surface, the bottom surface configured to be in contact with a patient's skin surface;
a first securing member connected to the top surface of the base, the first securing member comprising a first surface configured to receive the tubing of the vascular access device at a first location; and
a second securing member connected to the top surface of the base, the second securing member comprising a second surface configured to receive the tubing of the vascular access device at a second location, wherein the first securing member is positioned closer to the first side of the base than the second securing member.

14. The secondary securement device of claim 13, wherein the first surface and the second surface are each channels configured to receive the tubing of the vascular access device.

15. The secondary securement device of claim 14, wherein the first and second securing members are C-shaped.

16. The secondary securement device of claim 14, wherein the first surface faces the first side of the base and the second surface faces the second side of the base.

17. The secondary securement device of claim 13, wherein the first securing member is positioned closer to the first end of the base than the second securing member.

18. The secondary securement device of claim 13, wherein the base and the first and second securing members are formed integrally as one piece.

19. The secondary securement device of claim 13, further comprising a third securing member connected to the top surface of the base, the third securing member comprising a third surface configured to receive the tubing of the vascular access device at a third location.

20. The secondary securement device of claim 13, wherein the first and second securing members comprise an elastomeric material.
